(19) United States
(12) Patent Application Publication
(10) Pub. No.: US 2002/0058296 A1
(43) Pub. Date: May 16, 2002

(54) Contained indicators for determining sterilization

(76) Inventors: Michael J. Miller, Rochester, NY (US); Judy K. Hendricks, Albuquerque, NM (US)

Correspondence Address: BAKER & BOTTS, 30 ROCKEFELLER PLAZA, NEW YORK, NY, 10112

(21) Appl. No.: 09/951,068

(22) Filed: Sep. 13, 2001

Related U.S. Application Data
(63) Continuation of application No. 09/168,671, filed on Oct. 9, 1998.
(60) Provisional application No. 60/073,547, filed on Feb. 3, 1998.

(51) Int. Cl. C12Q 1/22; A61L 2/08; B29D 22/00

(52) U.S. Cl. 435/31; 422/26; 428/35.4

(57) ABSTRACT

This invention is directed to sterilization indicator systems for determining the effectiveness of sterilization processes. Test indicator devices comprise a container that is open on one end and has liquid impermeable and substantially gas non-absorptive walls which surround the biological or chemical material to be used as the indicator system, the opening contains a barrier that allows fluid such as gas to flow from the outside, through the plug and into the interior chamber containing the indicator system providing the detection. The entire indicator is further contained within a sealed pouch that is liquid impermeable and gas permeable under the conditions of a gas over steam sterilization system. The flexible pouch allows the test indicator to properly function while protecting the indicator from the environment while maintaining reliability of the test results. Although such sterilization indicators can be used in a wide variety of procedures, the indicators of the invention are particularly useful in evaluating the effectiveness of air overpressure sterilization.

### BACKGROUND OF THE INVENTION

1. Field of the Invention

This invention relates to improved indicator systems for determining the effectiveness of sterilization in which a test indicator device is placed in a pouch that is liquid impermeable and gas permeable under the conditions of a gas over steam sterilization system. The invention is also directed to methods of using the improved indicator systems. The indicator systems of the present invention are particularly useful in evaluating the efficacy of gas over steam sterilization.

2. Description of the Background

In health care, as well as many other industries, it is nearly always necessary to monitor the effectiveness of processes used to sterilize equipment such as medical devices, waste materials, instruments and other disposable or nondisposable articles. In these settings, sterilization is generally defined as the process of completely destroying all viable microorganisms including structures such as infectious viruses, spores, yeasts and fungus. The standard practice in hospitals is to include a sterility indicator in a batch of articles to be sterilized. The use of sterility indicators allows a direct and sensitive approach to assay the lethality of the sterilization process.

A standard type of biological sterility indicator includes a known quantity of test microbial spores. This indicator is placed into the sterilization chamber and exposed to the sterilization process along with the objects to be sterilized. The test microbial spores, for example Bacillus stearothermophilus or B. subtilis spores, are incubated for a specified period of time under conditions which favor proliferation. The exposed spores are then examined for indications of possible growth, such as turbidity in the growth medium or the presence or absence of certain metabolic products of any surviving microorganisms. Positive growth, indicating the presence of viable spores, indicates that the sterilization process was insufficient to destroy all of the microorganisms. While the apparatus for containing the spores has varied continuously, the general sterility detection process has not. Many such indicators as well as improvements thereon are disclosed in U.S. Pat. Nos. 3,239,429; 3,440,144; 4,596,773; 4,717,661; 4,732,850 and 5,167,923.

The largest use of sterility indicators occurs in research and the health care industry. Typically, such facilities have limited resources and must reuse their materials and instruments within 24 to 48 hours after sterilization and often immediately. Conventional sterility indicators normally require that the microorganisms be cultured for at least two and often up to seven days to assure adequate detection of any surviving microorganisms. During this time, items which go through the sterilization process should not be used until the results of the spore viability test have been determined. Consequently, a holding period for sterility verification is often required. This holding period is both impractical and inefficient and, thus, it is the major drawback of all conventional sterility indicators.

Conventional sterility indicators are typically viable spores which are exposed, along with the objects to be sterilized, to a sterilizing condition. After exposure, the indicator is removed and the spores are cultured under defined conditions. Culturing can take up to a full week for definitive results. The indicators often require post-sterilization incubation at higher than ambient temperatures to provide detectable results.

The use of an enzyme and its subsequent activity as an indicator in detecting sterility has been described in U.S. Pat. Nos. 5,073,488 and 5,272,488. This technology has been greatly advanced with U.S. Pat. No. 5,486,459, which describes the use of a plurality of interactive enzymes. This technique involves subjecting a set of interactive enzymes to a sterilization cycle. Following completion of the cycle, the set is incubated with a substrate which is acted upon by the enzymes and transformed into a detectable product. Enzyme-modified product can be detected, for example, calorimetrically or fluorometrically. This method has been proven to be accurate and detection speeds are greatly accelerated as compared to spore systems. In fact, definitive results using interactive enzyme technology can be determined in less than a few minutes. One such product, which is commercially available (North American Science Associates, Inc.; Northwood, Ohio), is the RSI® rapid indicator.

Sterility indicators including the RSI® are often placed in special packaging or wraps to simulate the condition of wrapped goods being processed in a sterilizer. If the articles to be sterilized are in special wrappings or packaging, the sterilant needs to effectively pass through the wrappings to destroy microorganisms on the article. To test the effectiveness of the sterilant passing through additional materials, sterility indicators are placed in challenge packs. These packs impede the sterilant as would the wrappings and thereby represent the conditions of wrapped goods in a sterilizer.

There are international standards such as the International Organization for Standardization (ISO) and the European Standards (EN) that deal with sterilization testing including steam sterilization. International standards dealing with biological indicators and testing procedures are found in the ISO 11138 series and EN 860 series. International standards for the air removal tests for pre-vacuum steam sterilizers comprising a chemical indicator in a test pack are found in the ISO 11140 series and EN 867 series. These packs incorporate the Bowie-Dick test and have similar performance standards as seen in AAMI (American Association of Medical Instrumentation), but use different testing procedures.

AAMI has proposed guidelines for challenge packs containing indicators that are assembled by hospital workers to simulate the conditions of wrapped goods in a steam or ethylene oxide sterilizer. Materials required for an AAMI challenge pack for a steam sterilizer include sixteen freshly laundered huck towels, autoclave tape and sterility indicators. In one method, each towel is folded length-wise into thirds and then folded width-wise in half. Towels are placed one on top of another with the folds opposite each other. Sterility indicators are placed between the eighth and ninth towels and the pack is secured with autoclave tape. The AAMI steam challenge pack is placed into a steam autoclave for the appropriate amount of time. Upon completion of a cycle, the indicators are processed to determine if the sterilization process was sufficient to inactivate the indicators buried in the pack.

In the case of ethylene oxide sterilization, AAMI recommends placing a sterility indicator into a plastic syringe so that the plunger is not touching the indicator. In this case, the needle end of the syringe is open. Two such syringes are placed in the center of a stack of folded towels and the stack is wrapped in a single towel. For routine monitoring, the syringe and indicator can be wrapped in a single towel and placed into a peel pouch.

Tests are also performed that evaluate the effectiveness of air removal in a prevacuum steam sterilizer. Prevacuum steam sterilizers are used to minimize the amount of air present in the sterilization chamber, thus enhancing the penetration of steam into porous loads. A prevacuum sterilizer air removal test is also known as the Bowie-Dick test or a prevacuum sterilizer residual air test.

AAMI guidelines for the Bowie-Dick test pack state that the standard pack is made using folded cotton surgical towels. Several towels are folded to create a stack 10 to 11 inches high with a rectangular border of 9 by 12 inches. A Bowie-Dick test sheet, which comprises a pattern of chemical indicator ink or indicator type on a porous sheet, is placed in the center of the pack. The pack is wrapped in a single cotton wrap and processed in a steam prevacuum sterilizer. The acceptance criterion is that the test sheet or tape darkens uniformly after processing. In other words, the chemical indicator ink changes color upon exposure to steam and if the entire sheet shows a uniform color change, there was no residual air to impede the steam.

AAMI guidelines state that other devices may be used in place of the AAMI challenge packs and Bowie-Dick tests if they provide equivalent results to the AAMI packs. Enclosure of sterilization indicators in various fibrous materials, analogous to textiles such as the towels used in the AAMI challenge packs, has been proposed in U.S. Pat. Nos. 5,200,147, 5,252,484 and 5,223,401. Packages in which a sterilization indicator is surrounded by porous material to replace some of the towels are described in U.S. Pat. No. 4,692,307.

There are several methods of heat sterilization that are commonly used in the art. The so-called gravity cycle sterilization process employs a saturated steam-vented system. Such a sterilization process is most applicable to products that can tolerate process temperature at saturated steam pressure. The gravity cycle sterilization process is commonly used, for example, on metallic products such as surgical instruments. Another method of heat sterilization is the vacuum cycle sterilization process, which employs saturated steam-forced air removal. This method is particularly applicable to the sterilization of porous materials and items having cavities where air is difficult to remove. The vacuum cycle is commonly used, for example, on towels, linens and other densely-packed surgical kits. Both the gravity cycle and the vacuum cycle are commonly used in hospital environments.

Another method of heat sterilization, the air pressurization system, also known as the gas over steam sterilization system or the air-overpressure sterilization system, employs an air-steam mixture cycle. This cycle is applicable to the sterilization of products in many industrial settings, especially for products comprising liquid fluids in a glass or plastic sealed container. This method is often utilized in industrial environments which include most of the manufacturing industry such as in packaging food and medical products, and in the manufacture of semiconductors. More specific industrial uses include terminal sterilization of contact lenses in blister packs or vials and the sterilization of intravenous (IV) bags for hospital use. Although this method of sterilization is relatively more complicated and, thus, more expensive than other procedures, it is still the accepted standard method of sterilization for these industries.

The air-steam mixture cycle utilizes an air-steam mixture cycle involving air overpressure during sterilization followed by temperature reduction while maintaining air overpressure and before venting. One characteristic of the air-steam mixture cycle is that liquid water is produced as a by-product. This water is generated on surfaces within the sterilizer and pools of water may form during the cycle. This water has the potential to interfere with the sterility indicator. Conventional sterilization detectors often fail because they are designed for hospital use. Modifications that might be necessary for industrial applications are simply not considered.

Although a number of test indicator devices have been developed, there is currently a need for an accurate and rapid sterility indicator that can function properly under adverse conditions, such as those created in the gas over steam sterilization system.

### SUMMARY OF THE INVENTION

The present invention overcomes the problems associated with existing test indicator designs and provides improved methods and apparatus for rapid and accurate evaluation of the effectiveness of the sterilization procedure, particularly those involving gas over steam sterilization systems.

One embodiment of the invention is directed to test indicator devices for determining the effectiveness of a sterilization procedure. These devices comprise a container having, preferably, liquid impermeable and substantially non-absorptive walls and at least one opening communicating with an interior chamber. The interior chamber contains biological material used as an indicator of the destruction of living organisms by the sterilization procedure and a fluid-transmissive barrier such as a plug positioned in the opening such that movement of fluid, preferably gas and not liquid, between the environment surrounding the test indicator and the interior chamber occurs through the fluid-transmissive barrier. The device is further contained in a sealed pouch that is substantially liquid impermeable and substantially gas permeable under conditions of the gas over steam sterilization system such that the sterilization effectiveness both outside and inside the pouch are substantially similar and preferably identical.

Another embodiment of the invention is directed to methods for determining the effectiveness of a sterilization procedure. These methods are preferably used in manufacturing products., and employ a test indicator of the invention.

Another embodiment of the invention is directed to methods for determining the effectiveness of a sterilization procedure for contact lenses in blister packs. These methods utilize test indicators of the invention in a gas over steam sterilization system by placing the test indicator inside a sealed pouch that is liquid impermeable and gas permeable under the conditions of gas over steam sterilization. The pouch may be nearly any size convenient and appropriate for holding test indicators and placing in the chamber of the system.

Other embodiments and advantages of the invention are set forth, in part, in the description which follows, and will be obvious from this description or may be learned from the practice of the invention.

### DESCRIPTION OF THE DRAWINGS

FIG. 1 Diagram of the construction of a container for a test indicator.

FIG. 2 An RSI® rapid indicator unit.

FIG. 3 Diagram of the operation of an enzyme-based test indicator such as the RSI® test indicator.

FIG. 4 Diagram of a challenge pack.

FIG. 5 A two container design challenge pack.

FIG. 6 RSI® test indicator challenge pack design.

### DESCRIPTION OF THE INVENTION

As embodied and broadly described herein, the present invention is directed to methods and apparatus for determining the effectiveness of sterilization in which a test indicator device is placed in a liquid impermeable, gas permeable sealed container.

Conventional sterilization indicators can be used to determine the effectiveness of a wide variety of sterilization processes. Many such processes create conditions during the sterilization cycle that damage the indicator. For example, certain sterilization cycles utilize large amounts of liquid which accumulates on surfaces and cavities in the sterilization chamber. Conventional test indicators should not be submerged in these liquids as liquids, especially water, can saturate the biological and/or chemical components of the detection or indication systems. This can produce adverse effects on the inactivation kinetics of these components causing inaccurate and incorrect readings.

It has been discovered that the effectiveness of a sterilization process can be determined by placing the test indicator within a pouch to protect the sterilization detection system from the conditions of the sterilization cycle. The pouch is preferably liquid impermeable and gas permeable under the conditions of a gas over steam sterilization system to protect the indicator. The sealed pouch allows the test indicator to properly function while protecting the test indicator from the environment. Further, or alternatively, test indicators of the invention may possess a gas-transmissive barrier over the opening containing the biological or chemical components. In this embodiment, the barrier is preferably composed of a hydrophilic material that will not become saturated with liquids such as water from the the sterilization cycle.

The pouch can be designed to be used with any sterilization indicator devices including, for example, the rapid indicator or RSI® (North American Science Associates, Inc.; Northwood, Ohio). The RSI® can be used generally for the rapid determination of the efficacy of different types of sterilization processes (e.g. steam heat, dry heat, chemical sterilant such as ethylene oxide, radiation, plasma). In one embodiment, the barrier is comprised of a compressible material placed as a plug into an opening or sleeve of a container, wherein the container or sleeve has a smaller cross-sectional area than the cross-section of the article when not compressed. Alternatively, the barrier may be a simple membrane such as, for example, a bacteria permeable or impermeable nylon or other polymer membrane. In any case, the barrier may be used to regulate the amount of sterilant (e.g steam, gas, chemicals or plasma) entering the test indicator. For example, with foam inserts, the amount and/or length of the foam insert utilized may be regulated according to the sterilizing process. Indicator reagents are placed in the container with at least one opening and the opening is filled with the compressed cylindrical insert.

Test indicators comprise an outer container having, preferably, liquid impermeable and gas non-absorptive walls, and at least one opening leading into a chamber which contains the sterilization detection system. Such systems include enzyme-based systems such as the RSI® which contains one or more components of an interactive enzyme system, spore-based or bacterial-based systems or simple chemicals. In any case, these biological and/or chemical components may be fixed to a solid support or free-floating in a non-aqueous or partially-aqueous solution. After sterilization, the user simply mixes the components in the container with other components that will initiate the detection system such as, for example, medium to initiate spore growth or enzyme substrate to initiate a chemical reaction. If spores are still present, the cycle was ineffective and there will be evidence of metabolic activity that can be detected such as enzyme activity or acid production. In pure enzyme systems such as the RSI®, if any enzyme activity is still present, substrate plus any necessary coenzymes, cofactors or other components are added to the exposed enzymes and interact to form detectable product which can be assayed to determine the effectiveness of the sterilization procedure. If the proper sterilization conditions were not met, the interactive enzyme system remains active, and a detectable product, which may be observed visually as a color, forms upon the addition of the remaining components of the enzyme system. If the proper sterilization conditions were met, the sterilant has destroyed components of the interactive enzyme system and no colored product is formed. Inactivation of the enzyme system parallels the inactivation of bacterial spores subjected to the sterilization process. Results using, for example the RSI® device, are available in from a few seconds to a few hours and preferably in less than one minute.

The container used in the test indicator device may be a vial or cylindrical tube in which the fluid-transmissible barrier is a foam stopper. That barrier may be gas- or liquid-transmissible and is preferably only gas-transmissible. The barrier can also act as a filter for fluids passing through the tube. In one embodiment, the barrier may be a compressible material which is a gas-permeable and bacteria-permeable, open-celled natural or artificial (plastic) foam or sponge comprised of, for example, polyurethane, polyester, polyether, cellulose, melamine or a combination of these materials. Foam density, pore size, cell structure (the percent of opened cells), size, shape, amount of foam, stiffness and tensile strength can be chosen to fit the particular situation.

For a container with a cross-sectional area of from about 0.03 to about 0.2 square inches, the compressed material has a non-compressed cross-sectional area of from about 0.2 to about 3.5 square inches. The inside portion of the plug is from about 0.4 to about 2.0 inches in length, preferably about 1.2 to 1.5 inches. The plug may also have an overhang portion which extends outside of the container. Preferably, the overhang portion is less than about 0.5 inches in length. These areas and lengths can be adjusted accordingly for larger and smaller sized containers. The inside length and/or the length of the overhang portion can be adjusted very easily during manufacture. Optimal lengths can be determined empirically by one of ordinary skill in the art according to the parameters of a particular sterilization process. Shorter lengths tend to be most useful for chemical process whereas longer plug and overhang lengths are typical for steam sterilization. Adjustments can also be made to the distance of the plug from the sensing system and the density, the degree of compactness and the composition of the plug. All of these factors affect the sensitivity of the indicator to the sterilization process.

The indicators are easily adjusted and can be modified to meet all major and minor alterations of a sterilization process. It is not necessary to switch to another type of sterilization indicator upon changing sterilization processes or sterilants. The barrier can be varied to optimize the sterility indicator and thereby meet multiple situations and different sterilants as well as different sterilization protocols. It is also not necessary to change the type of sterility indicator upon changing the sterilization process. Adjustments can be as simple as changing the barrier, and so it is a very straightforward matter to implement a change during manufacture with little to no added expense.

Sterility indicators systems may be spore-based or enzyme-based or based on a combination of both types of systems for providing an indication of sterility. Particularly useful are indicators utilizing interactive enzyme systems. Inactivation of the enzyme system by the sterilization process mimics the death of viable spores and provides nearly instantaneous and reproducible results. The reagents, whether spores, enzymes, enzyme systems or combinations, may be liquid or solid. Liquids are preferably in a non-aqueous or partially aqueous medium. Solids may be membranes such as disks and are preferably powders or tablets that contain granularized reagents. Such reagents can be made into a granulation by fluid-bed granulation. Fluid-bed granulation takes different components and co-immobilizes these components into clusters. Clusters comprise different components dried onto a seed particle. The granulation process begins by suspending a seed material in air and spraying a liquid material onto the seed. Other components are added either to the liquid solution or to the fluidized particles. Particles adhere to the liquid and form clusters of different components and, finally, moisture is removed from the clusters. The granulation process can be used to manufacture enzymes co-immobilized in a granulation or pressed into a tablet with little moisture as enzymes are typically most stable when packaged without water.

There are several ways two enzymes can be formed into a granulation product. For example, each enzyme can begin as a liquid solution. Using an inert solid seed material such as cellulose, one enzyme is sprayed onto the fluidized cellulose seeds. A second granulation is made of the second enzyme and the two granulations are blended together. Alternatively, the two enzymes could be mixed together as one liquid solution and sprayed onto the seed material. Alternatively, one or both enzymes could begin as a solid material. The solid material would be used as the seed material and a liquid binder solution is sprayed onto the seeds. Liquid solution is needed to create granulation and the solid, dry components adhere to the liquid solution. While the material is being fluidized, the high temperature and low humidity remove water from the granulation product and the enzymes are coimmobilized onto the seed material.

Granulations can also be pressed into tablets. For example, several granulations can be blended together using mechanical blenders and pressed into a single tablet. When working with several granulations, each can be tested for activity and then the final composition of the tablets activity can be adjusted by altering the amounts of each granulation component. The final enzyme tablet will contain very little water, typically less than about 5% and preferably less than about 3%.

Indicator reagents suitable for some applications comprise a single enzyme, such as that described in U.S. Pat. No. 5,073,488, along with the substrates, reagents, catalysts, co-factors, etc., necessary to produce a detectable product. Indicator reagents may also comprise multiple components of an interactive enzyme system. For use with the invention, the enzyme system preferably comprises a known mix of enzymes, coenzyme, catalysts, cofactors, substrates, other reaction reagents or combinations thereof, such as those provided in U.S. Pat. No. 5,486,459. Enzyme systems comprise a plurality of enzymes that rapidly catalyze a series of coupled reactions which together produce a detectable product.

As noted above, one method of sterilizing is known as a gas over steam sterilization system. Such systems utilize an air-steam mixture cycle involving air overpressure during sterilization followed by temperature reduction while maintaining air overpressure and before venting. The air-steam mixture cycle is applicable to the sterilization of products in many industrial settings, especially for products comprising liquid fluids in a sealed glass or plastic container. Examples from the food industry include apple sauce, jams and jellies, cold cuts and other prepared meats, beverages, cheeses and many others. Other industrial uses include, for example, the terminal sterilization of contact lenses in blister packs or vials and the sterilization of intravenous (IV) bags for hospital use.

A relevant characteristic of the air-steam mixture cycle is that liquid water is produced as a by-product, and pools of water form in areas of the chamber as well as on the product being sterilized during the cycle. This water when generated on surfaces of the product transfers the latent heat from the steam to the load. When conventional test indicator are used as sterilization detectors, they often become submerged within the pools of water. Accordingly, test indicators of the invention preferably contain only a gas-transmissive barrier, wherein the barrier is not transmissive to liquids. In addition, it is also preferable that the barrier be composed of a hydrophobic substance that does not become saturated with liquid water such as a plastic or other polymer. As discussed above, saturation could have an adverse effect on the inactivation kinetics of the biological material used in the test indicator.

To prevent interference of liquid with the indicator, the test indicator is placed inside a pouch. The pouch may be of any size suitable for containing the test unit and for placing in the sterilization chamber. It may be rigid or flexible to be placed, for example, attached to a wall of the chamber or a particular object being sterilized. The pouch is preferably gas permeable (which gas includes water vapor used as sterilant), but liquid impermeable, that is, liquid sealed under conditions of air-over-pressure sterilization. It has been surprisingly found that such liquid impermeable-gas/vapor permeable pouches allow the test indicator to properly function while protecting the test indicator from the environment, namely steam under pressure that otherwise would prevent the proper functioning of the test indicator. In particular, the pouch is used to protect the device from liquid water when using an air-steam mixture cycle, thereby maintaining the reliability of the test results. This embodiment can be used to monitor sterilization as described herein.

Sealable pouches that are selectively permeable are conventionally known and commercially available. The seals of the pouch package must be strong enough to withstand steam at 270° F. under pressure yet be easy to open, preferably peel open easily and cleanly and deliver the test indicator for further evaluation. Package types include flat pouches, vent bags and gussetted pouches. Preferably, the pouch is a flat package comprising, on one side, a plastic sheet that is clear transparency for optical viewing and, on the other side, a paper sheet side that is vapor permeable and optimized for permeability of the sterilant. The vapor impermeable plastic may be heat sealed to the paper. Suitable plastic materials include, for example, polypropylene, polyamides such as nylon, polyester, polyvinyl chloride and HDPE, and copolymers an combinations thereof. The package may also be composed of two or more plastics. Suitable vapor permeable materials include, but are not limited to, paper and fibrous plastics such as Tyvek® sheet material. The plastic can be heat sealed to the paper. For example, a self-sealing sterilization pouch is available in various sizes from Cross Country Paper Products Corp. (Hauppuge, N.Y.) under the trademark Crosstex®. Preferably, the pouch is 3.5 by 5.25 inches or smaller, which is sufficient to contain a rapid sterility indicator unit as described herein, without taking valuable space within the sterilization chamber.

Accordingly, one embodiment of the invention is directed to a test indicator device comprising a container with at least one opening containing one or more spores, one or more enzymes, one or more enzyme systems (e.g. multiple-interacting enzymes or a plurality of components of an enzymatic process) or combinations thereof. The container may possess a barrier such as a cap, plug or membrane over the container opening which may be a screw cap, a snap cap, a fixed cap, a cap containing an opening that is smaller than the container opening, or may be open to the environment (i.e. no cap). For certain uses, the container may be placed inside a pouch such as, for example, a flexible or rigid plastic pouch to protect the container from the environment and from any damage by objects in the sterilization chamber. The pouch is preferably gas permeable and liquid impermeable or only semi-permeable, but may be made permeable or impermeable to any particular element (e.g. gas, liquid, solid) as appropriate for a particular use or as desired. For example, such a pouch can be used to protect the test indicator from interaction with liquid water when using the gas over steam sterilization system. The pouch can be used to protect the device from any other environmental parameter by using a pouch that is not permeable or only slightly permeable to the detrimental parameter thereby protecting the device and maintaining the reliability of the test results. Pouches that are selectively permeable are conventionally known and commercially available. These devices can be used, for example. to monitor sterilization as described herein.

Another embodiment is directed to a test indicator device for determining the effectiveness of a sterilization procedure comprising a container having liquid impermeable and substantially non-absorptive walls and at least one opening communicating with an interior chamber, the chamber containing biological material used as an indicator of the destruction of living organisms by the sterilization procedure and a gas-transmissive plug positioned in the opening such that movement of gas between the environment surrounding the test indicator and the interior chamber occurs through the gas-transmissive barrier, further comprising surrounding the container by a sealed pouch that is liquid impermeable and gas permeable under the conditions of a gas over steam sterilization system.

In a preferred embodiment the pouch comprises a gas-permeable portion and a gas-impermeable portion. The gas-permeable portion may be made of paper or a fibrous plastic sheet. The gas-impermeable portion may be made of a clearly transparent plastic film. The pouch may be adapted to peel open to deliver the test indicator for hands-on evaluation. The plastic may be selected from polypropylene or the group consisting of nylon, polyester, polyvinyl chloride, and high-density polyethylene or copolymers and combinations thereof.

Another embodiment of the invention is directed to methods for determining the effectiveness of a sterilization procedure for manufactured products. According to these methods, a test indicator is placed into a flexible pouch, which may be liquid impermeable and gas permeable, for determining the effectiveness of a gas over steam sterilization system. The pouch may comprise a gas-permeable portion and a gas-impermeable portion. The gas-permeable portion may be made of a paper or a fibrous plastic sheet. The gas-impermeable portion may be made of plastic. The pouch may be peeled open to deliver the test indicator for hands-on-evaluation. The manufactured products being sterilized may comprise liquid fluids in sealed glass or plastic containers. The products may further be selected from the group consisting of contact lenses in blister packs, IV bags and single-use food packets. The products may be adhesively sealed in plastic containers that might otherwise expand or open under heat without external pressure.

Still another embodiment is directed to a method for determining the effectiveness of a sterilization procedure for contact lenses in blister packs. These methods employ a test indicator comprising a container having liquid impermeable and substantially non-absorptive walls and at least one opening communicating with an interior chamber, the chamber containing biological material used as an indicator of the destruction of living microorganisms by the sterilization procedure and a gas-transmissive plug positioned in the opening such that movement of gas between the environment surrounding the test indicator and the interior chamber occurs through the gas-transmissive plug. The test indicator is further contained within a sealed pouch that is liquid impermeable and gas permeable under the conditions of gas over steam sterilization.

Still another embodiment of the invention involves the sterilization under pressure of adhesively sealed liquid fluids in plastic containers that might otherwise expand and open under heat without external pressure. Such products cannot be safely sterilized by sterilization processes that employ either a gravity cycle or a vacuum cycle.

The invention is designed to determine the effectiveness of the sterilization process, and is particularly useful in a gas over steam process. Effectiveness may be determined by detecting or monitoring the sterilization process. The invention is preferably used in conjunction with a basic process that subjects at least one, and preferably multiple, components of an enzyme system to the sterilization procedure. The enzyme system comprises a known mix of enzymes, coenzymes, catalysts, cofactors, substrates, other reaction reagents or combinations thereof, which is housed in a test indicator. The components have an interdependent activity which correlates with the viability of the microorganisms used in state-of-the-art biological indicators.

Indicators comprised of enzymes and preferably interactive enzyme systems are a suitable substitute for spores. Inactivation of an enzyme system by a sterilization process mimics the death of viable spores. In all cases, the results that can be achieved are rapid as well as reliable and reproducible.

One preferred embodiment of the invention comprises a test indicator such as the RSI® which is placed into a pouch to be subjected the sterilization process. After the sterilization cycle is complete, components of the detection system are added to form a mixture. The mixture is incubated, if necessary, for a period of time sufficient to allow for product formation from the interaction of the enzymes with the substrate. Incubation times range from a few seconds to minutes and are preferably less than about 15 minutes, more preferably less than about 10 minutes and even more preferably less than about 3 minutes. If desirable, incubation can be eliminated and the product detected almost immediately or in less than about 20 seconds. A detectable product will form if all of the components of the enzyme system, including the plurality of enzymes, are present and active. A positive result is observed when each exposed component survives denaturation and is able to function interactively to produce a detectable enzyme-modified product. The enzyme-modified product as an indicator of residual activity is visually detectable within 1 to 60 minutes and preferably within seconds. Any change detected, which is preferably a color change, is an indication to an observer that the sterilization cycle had not inactivated certain components and, thus, was insufficient to assure sterilization of other articles exposed to the sterilization procedure. Conversely, an absence of a color change indicates that the sterilization procedure had inactivated at least one of the components thereby preventing the interactive reaction from taking place and thus, an equivalent of rapidly and directly detecting the survivability of bacterial spores in a similar conventional test.

Lack of detectable enzyme-modified product within the established period of time indicates a sterilization cycle which has been lethal to the function of the interactive enzyme system as well as lethal to a viable 10⁶ population of Bacillus stearothermophilus spores. Generally, these values are expressed as D-values, which is the time taken at a given temperature to reduce the viable population of test microorganisms to ten percent of its original value. Inactivation of the enzyme system parallels the inactivation of bacterial spores subjected to the sterilization process, except that the result may be available in minutes or seconds as compared to at least overnight incubation required for detection of bacterial growth from spores.

The ability of the components of an enzyme system to survive conditions which only partially kill test microorganisms is dependent, at least in part, upon the use of a barrier between the sterilant and the enzymes, and that the interactive enzyme system will remain active following a sterilization cycle which is insufficient to kill the test microorganisms. It is not necessary that the barrier be impermeable to microorganisms such as bacteria, only that it be fluid permeable to permit exposure of the indicator components to the sterilizing environment, such as through open cells of a compressible material or around the sides of a closed cell compressible material. This provides a direct correlation of spore viability with the interactive activity of the enzymes of the system which, following an inadequate sterilization cycle, is sufficient to convert a substrate system for those enzymes to a visually detectable concentration of product within a relatively short time, preferably 1 to 60 minutes. The basis for the correlation between the activity of the enzymes and other components to the germination and growth of microorganisms is due to the commonality of both in their reliance upon systems of biologically derived interacting enzymes and coenzymes to function. The sterility indicator demonstrates that there is a direct correlation between the conditions to kill a microorganism and the conditions to inactivate a component of a network of interacting enzymes. In fact, the interactive system can be considered to mimic a bacterial spore in that there is a semi-permeable membrane, the spore wall, that encases a collection of interactive enzymes. In the case of an amplification interactive enzyme system, if any one of the key enzymes, coenzymes, cofactors, substrates, catalysts, or other reagent components of the system are totally inactivated when an indicator solution is added, no color change will occur, thus, mimicking conventional spore systems, but able to provide results at much faster speeds.

Using the test indicators of this system, sterility verification is determined from completion of the test results which, surprisingly, can be very rapidly achieved because the reliability of conventional biological indicators is combined with the speed of techniques closer to that utilized by enzymatic and chemical indicators. Further, unlike spores, resistance is correlated with activity, and in enzyme systems containing enzymes, coenzymes, catalysts, substrates or other reagents of an interactive system, stability can be very precisely quantitated individually as well as in multiple enzyme systems. Therefore, use of interactive enzyme systems not only increases speed, but a level of standardization which is far superior to that obtained with conventional biological or other enzymatic techniques can be achieved.

Although most useful in gas over steam procedures, the invention can be used with adjustable indicator systems for the determination of the effectiveness of sterilization processes using steam, gas, radiation, chemical and plasma sterilizers, which are used in many hospitals, laboratories, and clinics, as well as in research institutions, in food and environmental laboratories, and in all technologies which utilize sterilization in manufacturing, production or waste disposal.

Sensitivity of sterility indicators can be adjusted quickly and easily for the manufacture of sterility indicators reactive to one or more predetermined parameters. For example, a test indicator substantially identical to the sterility indicator is exposed to a sterilization procedure and the effectiveness of that test indicator for reacting to the predetermined environmental parameter determined. The position of the test indicator within the pouch can be adjusted so that the indicator remains at one location and a variety of locations tested for accuracy and reliability. From the results determined for each test indicator, the sensitivity of the indicator can be optimized for detection to the specific environmental condition or conditions.

When used with the present invention, the sterility indicator preferably includes a biologically relevant material, such as bacterial spores or preferably a source of multiple interacting enzymes with the gas-transmissive barrier between the components and the opening. Interacting components are preferably localized within close proximity to one another such as within the matrix of a cellulose filter disk or granulation product, and/or within a defined medium and are thus, co-immobilized. One or more enzymes, substrates, coenzymes or catalysts may be included on the solid matrix. Within the container is an effective amount of a gas-transmissive material to form the barrier which is semi-permeable to the transmission of liquids and gases, and an effective means for maintaining a finite distance between the semi-permeable opening and the enzymes. The barrier may be liquid permeable or impermeable which reduces the likelihood of slippage that may sometimes occur with plungers and stoppers. Also preferable is a barrier which is a plug that is constructed of a polymer such as a synthetic, a plastic, a rubber, Gore-Tex (a gas transmissive and liquid impermeable polymer) or a combination thereof. A Gore-Tex barrier would be liquid impermeable whereas an open cell foam barrier, such as a sponge, would be liquid semi-permeable.

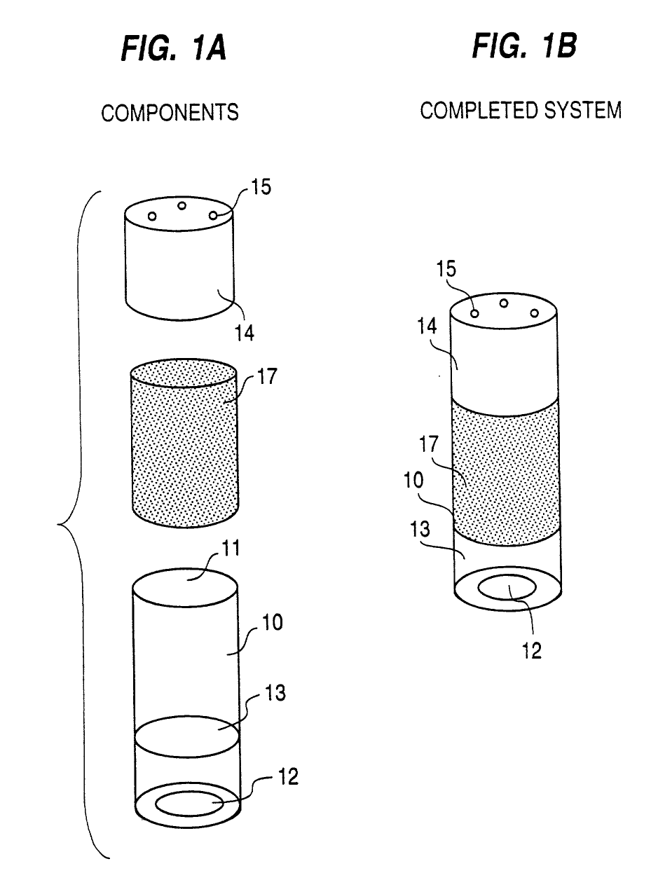

One rapid multiple enzyme sterility indicator which may be used in the present invention is illustrated in FIG. 1. The indicator comprises cylindrical tube 10 having liquid impermeable walls with single opening 11 at one end. Cylindrical tube 10 contains solid support disk 12 upon which multiple interacting enzymes are co-immobilized. Cylindrical tube 10 also contains non-aqueous medium 13 covering solid support disk 12. Single opening 11 is covered with cap 14 having a plurality of holes 15 allowing unimpeded access of sterilant through single opening 11. The apparatus of FIG. 1 is assembled by placing solid support disk 12, upon which multiple interacting enzymes are co-immobilized, into the bottom of cylindrical tube 10. Non-aqueous medium 13 is added to cover solid support disk 12. A cylinder of heat resistant foam material 17 is compressed into cylindrical tube 10 providing a structural framework for the containment of non-aqueous medium 13. Foam material 17 also serves to maintain a fixed distance between the multiple interacting enzymes co-immobilized upon solid support disk 12 and single opening 11. Cap 14 is placed on top of cylindrical tube 10 covering single opening 11.

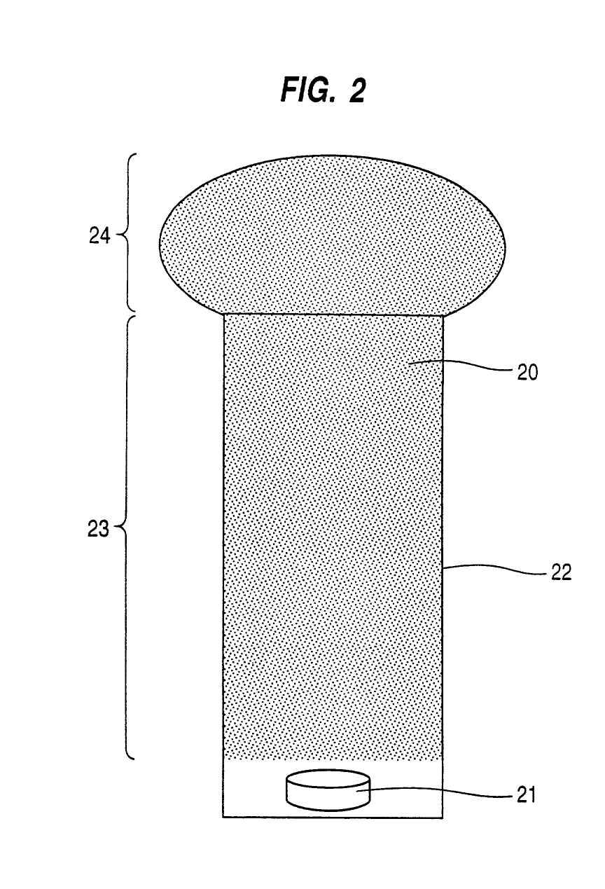

A preferred indicator unit for use in the present invention is the rapid-multienzyme sterility indicator shown in FIG. 2. This multiple-enzyme sterility indicator comprises a test unit and indicator solution. The test unit is comprised of cylinder tube 22 having liquid impermeable walls with an opening at one end. Cylindrical tube 22 contains granulized tablet 21 comprising the co-immobilized interacting enzymes. The opening of the tube is filled with compressed foam insert 20. The foam material regulates the amount of sterilant reaching the tablet containing the interacting enzymes.

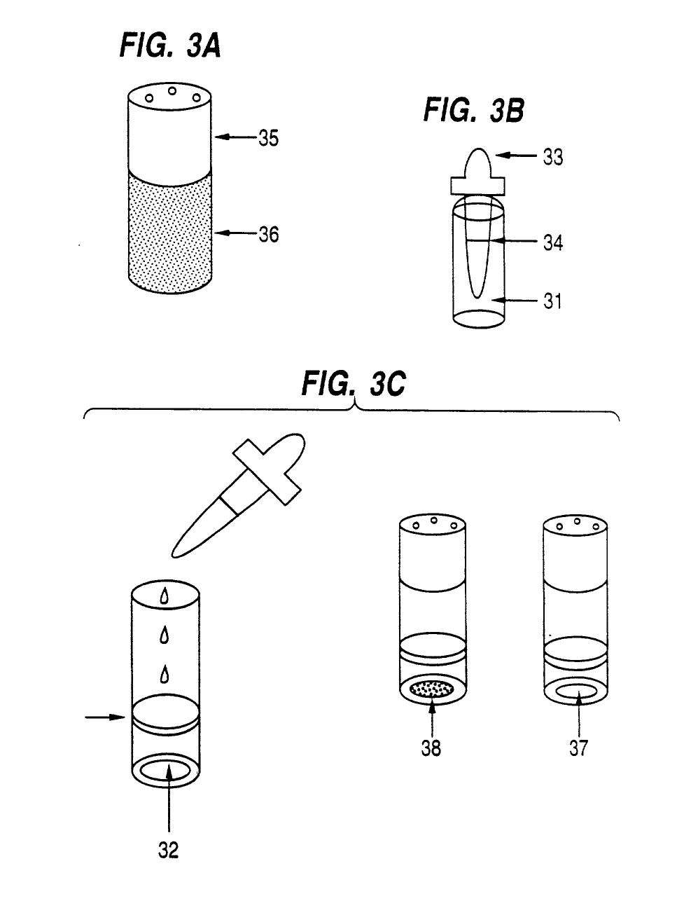

The dispenser of the indicator solution is shown in FIG. 3. A bottle contains indicator solution 31 which produces a visual color change when added to active multiple interacting enzymes co-immobilized on solid support disk 32. The bottle contains eyedropper 33 with premeasured volume line 34. Filling eyedropper 33 to premeasured volume line 34 with indicator solution 31 assures that the correct volume or number of drops of solution, is dispensed into the tube.

A method for conducting the sterility test is also illustrated in FIG. 3. The pouch containing the sterility indicator is placed into the sterilizer along with other materials which are to be sterilized and is exposed to the sterilant during the course of a sterilization cycle. After the completion of the sterilization cycle, the sterility indicator is removed from the sterilizer and pouch and allowed to cool to room temperature. Cap 35 and foam material 36 are removed and can be safely discarded. Indicator solution 31 is drawn into eyedropper 33 using the premeasured volume line 34 to assure that the correct volume of indicator solution is used and dispensed into the tube. The resulting mixture is incubated, if necessary, at room temperature for seconds to minutes, preferably for less than about 10 minutes and more preferably for less than about 3 minutes. The solid support disk is visually inspected at the end of the incubation period. An absence of red coloration on the solid support disk (e.g., white) indicates negative result 37 and signifies a successful sterilization cycle. The presence of red coloration on the solid support disk indicates positive result 38 and signifies an unsuccessful sterilization cycle.

The indicator in the pouch of the present invention is useful in evaluating gas over steam procedures. However, the indicator may also be used for autoclaving (121° C. or higher, such as 132° C. or 134° C.), a chemical procedure utilizing ethylene oxide or another appropriately lethal chemical or dry heat of temperatures between about 50° C. to about 200° C., radiation, or a plasma-phase sterilization procedure. These procedures are practiced in the health care industry, but also in industries having to do with environmental technology, food manufacturing, waste disposal and in those technologies where sterility is required.

The indicators described herein can be used to determine the effectiveness of a sterilant to pass through a tortuous path such as a challenge pack. Challenge pack testing, can make use of the same design and the same adjustable features. An AAMI steam challenge pack comprises a biological indicator such as bacterial spores on an inert carrier, wrapped in 16 surgical towels. The towels create a tortuous path for the steam to reach the indicator. This simulates the wrapped goods processed in a steam sterilizer in a hospital setting.

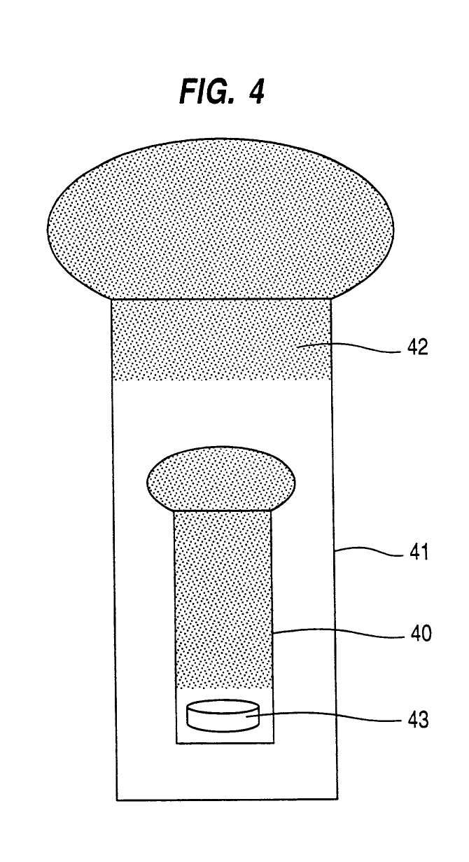

Challenge packs are used to test the effectiveness of the sterilant to pass through the pack and reach the indicator. This simulates wrapped goods processed in the sterilizer. The foam insert design can be used for challenge pack testing. A sterility indicator, either the enzyme-based indicator described previously or a conventional spore-based indicator may be used in this type of challenge pack. An example of a challenge pack is shown in FIG. 4.

Sterility indicator 40 is placed into container 41 which has at least one opening filled with a predetermined amount of foam 42. Container 41 has substantially gas non-adsorptive walls so the sterilant has to enter through foam 42 to reach sterility indicator 40 and thereby regulates the amount of sterilant entering the container. Sterility indicator 40 contains either spores or enzymes 43. Foam 42 regulates the amount of steam or sterilant entering the container. After the challenge pack has been exposed to the sterilization process, the indicator is removed from the challenge pack and processed. If the indicator is positive, proper sterilization conditions were not achieved within the pack. A negative result means proper conditions were met. The rapid sterility indicator described above or a standard biological indicator can be used in conjunction with the challenge pack. The challenge pack is simple to use and provides reproducible results. The desired amount of challenge can be easily reproduced to mimic the challenge described by standards such as AAMI, ISO or EN for a steam or ethylene oxide challenge pack.

Indicators employing the foam insert design may be used for the air removal test. The air removal test comprises a container with a Bowie-Dick test sheet or a chemical indicator on a carrier. The transparent container comprises at least one opening which is filled with foam. After the test cycle is complete, the air removal test is removed from the sterilization chamber. The user observes the uniformity of the color change of the chemical indicator. Since the material of the container is transparent, the user would simply observe the uniformity of the chemical indicator ink. Thus, there is no need to unwrap the device.

The air removal test is also based on the similar design. By placing a chemical indicator into a transparent container with a foam insert, the prevacuum air removal test equivalent is made. The air removal test is placed into a prevacuum steam sterilizer. After the cycle is complete, the user can simply view the uniformity of the color change of the chemical indicator in the transparent container or simply open the container and remove the chemical indicator.

The foam insert design overcomes many disadvantages of the current designs for testing the effectiveness of sterilization processes. The foam insert design can be used as a component of a rapid sterility indicator composed of interactive enzyme systems which can provide nearly instantaneous results. A sterility indicator with the foam insert design also offers the advantage of being adjustable to suit various types of sterilization process. Presence of foam also allows the sterility indicator to effectively control the amount of sterilant entering the device in a standardized manner. The enzyme content of the rapid sterility indicator and the foam specifications can be easily controlled to provide reproducible results during manufacturing. Conventional biological indicators that are based on the inherent resistance of bacterial spores can not be as easily controlled.

The foam insert design also overcomes disadvantages of challenge pack and air removal test designs. Assembly of AAMI test packs is very time consuming. AAMI packs are not standardized in the sense that differences in how individuals make the packs and different types of towels can result in packs with differing characteristics. The advantages of the foam insert design is that it can be used for a sterility indicator for many types of sterilizers as well as challenge packs and air removal tests, it is simple to use and it is standardized and reproducible. The fact that the same design can be used for multiple tests (e.g. sterility, challenge packs, air removal) offers simplicity to the users. The challenge pack and air removal test designs also allow the user to quickly and easily retrieve the indicator. There is no need to unwrap many towels to retrieve the indicator. The transparent container also permits the user to confirm that an indicator is present in the pack.

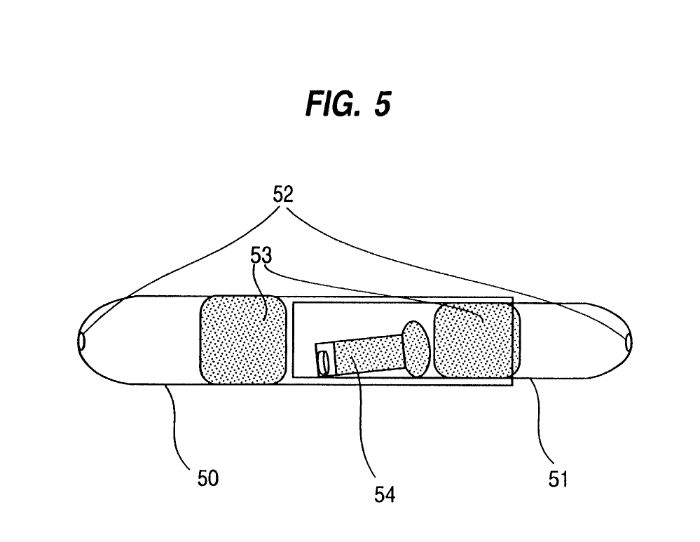

Another embodiment of the invention is directed to a method for determining the effectiveness of a sterilant to pass through a tortuous path comprises a challenge pack that employs the foam and container design that allows the users to easily open and close the challenge pack for repeated use. The challenge pack comprises two containers, two foam inserts and a sterility indicator, as shown in FIG. 5. Each container has at least two openings, one opening is the same size as the diameter of the container, the second opening is much smaller. One container 50 has a slightly larger diameter than the other container 51. Each container has a small hole 52 opposite the larger opening and a piece of foam 53 placed near the small opening. A sterility indicator 54 is placed into the smaller diameter container and the second container with a slightly larger diameter is placed over the first container's large opening. The containers fit together tightly due to their similar diameters. Instead of relying on the similar diameters to hold the two tubes together, a screw closure, a snap locking device or a twist locking device could be used. The containers have substantially gas non-adsorptive walls so that the sterilant has to enter through small holes 52 and pass through the foam inserts 53 to reach sterility indicator 54.

After the challenge pack has been exposed to the sterilization process, the two containers are dislodged from one another and the indicator is retrieved and processed as usual. This test pack can be used again by placing an unexposed indicator into one of the containers and replacing the second container over the first container as shown in FIG. 5. The sterility indicator can be the enzyme-based indicator or a conventional spore-based indicator.

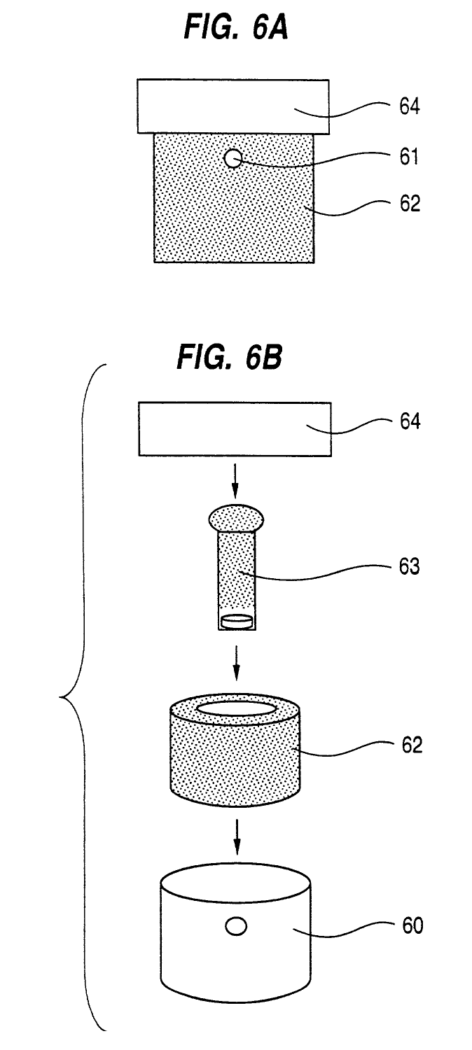

Another embodiment is a reusable challenge pack design which comprises a single container with a closure device, foam and a test indicator as shown in FIG. 6. The bottom portion of cylindrical container 60 has two holes 61 in the sides of the container. Tubular foam insert 62 fits tightly into the container. Foam insert 62 has a hole in the center which conforms to the shape of sterility indicator 63 which fits tightly into foam insert 62. Screw cap 64 is placed over the large opening of the container. When screw cap 64 is secured onto the container containing sterility indicator 63, sterilant would pass through the small openings in the sides of the container and through foam insert 62, before reaching sterility indicator 63. This is a tortuous path for the sterilant. This design would perform equivalent to other challenge packs. The materials are a non-absorptive plastic which can withstand multiple exposures to sterilization. The sterility indicator can be the enzyme-based indicator or a conventional spore-based indicator. By using a larger container and corresponding larger foam insert and replacing a chemical indicator (test sheet covered with unexposed chemical indicator ink) for the sterility indicator, this design could be used for air removal tests in pre-vacuum sterilizers. After processing in a pre-vacuum sterilizer, the uniformity of the chemical indicator color change would be used to determine if any air was present in the chamber. If air was present, the color change of the chemical indicator would not be uniform.

The following examples illustrate embodiments of the invention, but should not be viewed as limiting the scope of the invention.

### EXAMPLES

### Example 1

### Rapid Sterility Indicator for Monitoring Steam Sterilization Process

A rapid sterility indicator for steam sterilization is composed of a test unit and indicator solution. The test unit comprises a cylindrical glass vial, a tablet containing components of a plurality of interacting enzyme systems, a foam insert, and a label. The glass vial is approximately ¼ inches in diameter and 1 inch high, open on one end.

The tablet, containing two interactive enzymes of the enzyme system, is contained within the vial. The tablet contains a granulation of glucose dehydrogenase and diaphorase. The preferred enzyme concentration for each enzyme is 8 to 15 units per 20 mg tablet. The opening of the vial is filled with a compressed foam insert which is preferably a cylinder, with a diameter range of ¼ to 1 inch, preferably approximately ½ inch and the length range ⅛ to 3 inches preferably approximately 1½ inches in length. Foam is partially open celled with a density of approximately 6 pounds per cubic foot and the foam material is polyurethane. The detailed specifications included: polyester foam, open cell, 6 lbs, charcoal color, density was 5.00-6.60 p.c.f., strength 20.0-40.0 p.s.i., elongation 300-500%, tear resistance was 3.0-5.0 p.l.i., compress set 3.0 to 10.0%, load defection, 0.50 to 0.90 p.s.i., flammability HF-1, and cell size 50-70 c.d.i. On the outside of the vial is a label containing steam-sensitive indicator ink.

This test unit was placed into the sterilization chamber of a steam sterilizer operating at 121° C. along with the items to be sterilized. After the cycle was completed, the test unit was removed from the sterilization chamber. The color change of the steam-sensitive indicator ink served to identify the processed units from unprocessed units. The foam insert was removed and five drops of the clear, colorless indicator solution were added to the vial containing the white enzyme tablet. The indicator solution was packaged in an amber glass bottle with a dropper dispenser. The indicator solution contained p-iodonitrotetrazolium violet within a range of 32 μM to 16 mM, preferably 3.2 mM; NAD (β-nicotinamide adenine dinucleotide) within a range of 1 μM to 5.5 mM, preferably 0.11 mM; glucose within a range of 1% to 90% preferably 10%; ethanol within a range of 1% to 95% (by volume), preferably 5.5%; citric acid within a range of 0.0032 mm to 3.2 m, preferably 17 mm. The preferred buffer was 0.05 M Tris, pH 6.0-8.5.

The predetermined survival cycle in a BIER vessel operating at 121° C. was 5 minutes, the kill cycle was 15 minutes for sterility indicators. Survival cycles were cycles with short exposure times in which indicators should test positive, indicating that proper sterilization conditions were not met. Kill cycles were usually the standard cycles times in which the indicator should test negative indicating proper sterilization conditions were met. After indicator solution was added to the white tablet, the color of the tablet was visually observed for 1 to 20 seconds. After a 5 minute autoclave survival cycle at 121° C., which represented inadequate sterilization conditions, a positive result was expected and observed. in which the enzymes were active and a red colored product was formed on the surface of the tablet at or before 20 seconds. After a 15 minute autoclave kill cycle at 121° C. which represented an adequate sterilization cycle, a negative result was expected and observed, in which one or more enzymes were inactivated and no red colored product was formed. These positive and negative results paralleled the results of bacterial spores exposed to similar conditions in a steam autoclave.

TABLE 1
Steam Sterilization Test in a BIER Vessel

| | Survival Time | Kill Time |
|---|---|---|
| | (5 Minutes) | (15 Minutes) |
| Rapid Sterility Indicator | 10/10 | 0/10 |
| Sportrol spore strips | 10/10 | 0/10 |
| (10⁶ B. stearothermophilus) | | |

Table 1 shows results from a typical experiment. Results were recorded as the number of positives over the number tested (121° C.) in a BIER vessel. Rapid sterility indicators provided both positive results after the survival cycles and negative results after the kill cycles. These results demonstrate at least the equivalence of rapid sterility indicators to conventional biological indicators.

### Example 2

### Foam Structures to Mimic AAMI Challenge Packs

AAMI challenge packs provide a tortuous path for steam penetration. An analogous challenge pack for a steam sterilizer can be created using a container, foam, and a rapid sterility indicator (as described in Example 1) or a biological indicator. The challenge pack container is a plastic or glass tube, preferably plastic, measuring approximately 1.125 inch diameter and 5 inches in length with one opening in the container. The container contains a heat sink material such as a metal object. In this case a rapid sterility indicator test unit, as described in Example 1, is placed into the container. The one opening of the container is filled with a foam insert. Foam insert is approximately 2 to 4 inches in diameter and 1 to 4 inches long (non-compressed measurements). Foam is partially open celled with a density of approximately 1 to 6 pounds per cubic foot and foam material is preferably polyurethane.

Challenge packs were placed into a steam sterilizer operating at 121° C. or 134° C. and exposed to the predetermined survival and kill time intervals. Survival cycles are cycles with short exposure times in which indicators should test positive, indicating proper sterilization conditions were not met. Kill cycles are the standard cycles times in which the indicator should test negative indicating proper sterilization conditions were met. The rapid sterility indicators were processed, as described in Example 1, and results were obtained at 20 seconds or less after adding indicator solution.

The AAMI challenge pack was constructed and tested for comparison. The materials required for a AAMI challenge pack for a steam sterilizer were 16 freshly laundered huck towels, autoclave tape and sterility indicators. Each towel was folded lengthwise into thirds and then folded width-wise in half. Towels were placed one on top of another with the folds opposite each other. Rapid sterility indicators and conventional biological indicators were placed between the eighth and ninth towels. The pack was secured with autoclave tape. The AAMI steam challenge packs were placed into a steam autoclave at 121° C. for the appropriate amount of time. After the cycle, indicators were processed as in Example 1.

TABLE 2
Challenge Pack Analog
Exposure time to 121° C. steam

| | Survival Time | Kill Time |
|---|---|---|
| | (10 Minutes) | (30 Minutes) |
| Rapid Sterility Indicators in | 6/6 | 0/6 |
| Foam Challenge Packs | | |
| Rapid Sterility Indicators in | 6/6 | 0/6 |
| AAMI Challenge Packs | | |
| Sportrol spore strips | 6/6 | 0/6 |
| (10⁶ B. stearothermophilus) | | |
| in AAMI Challenge Packs | | |

The results in Table 2 were recorded as the number of positives over the number tested. As shown, when exposed to the survival cycle time, all indicators tested positive. When exposed to the kill cycle time, all indicators tested negative. This table also shows the equivalence of the conventional biological indicators in AAMI challenge packs to the rapid sterility indicators in AAMI challenge packs. These results demonstrate the equivalence of the rapid sterility indicator in the foam challenge pack to the AAMI steam challenge pack.

### Example 3

### A Single Container Reusable Pack Challenge Packs with Unique High Resistant Parameters

Challenge packs can be modified to create an extremely resistant challenge. By increasing the foam density and/or making the steam entry holes smaller, the challenge pack will enable the indicator inside the pack to survive very long exposures to sterilization. A reusable challenge pack can be created from one container with a closure device, one piece of foam and a sterility indicator. A reusable challenge pack is made of material that can withstand multiple exposures to sterilization and can be easily opened and closed. The container is made from plastic or glass, preferably plastic. The container is approximately 1½″ diameter, 2½″ long with a screw cap. The two steam entry holes are approximately ⅛″ in diameter and the holes are located approximately ¼″ below the cap. A tubular piece of open cell, 4-6 pound polyurethane foam approximately 1½″ long, 1½″ outer diameter (inner diameter ⅜″) is placed into the container. The rapid sterility indicator test unit as described in Example 1 is placed into the foam insert. The foam fits tightly into the container and the test unit fits tightly into the foam. The cap of the container is screwed into place and creates a tight seal so that the only pathway for steam to enter the challenge pack is through the small openings, passing through the foam to reach the indicator. The pack may also contain a heat sink material such as a metal object.

These challenge packs were placed into a steam sterilizer operating at 134° C. (pre-vac sterilizer) and exposed to the predetermined survival and kill cycles. For this high resistant challenge pack the survival cycle included exposure to 4 pre-vacuum cycles of temperature range of 60-130° C. for 8 minutes, then 1 minute of exposure to 134° C. The kill cycle included exposure to the 4 pre-vacuum cycles and a 4 minute exposure to 134° C. After the kill cycle indicators should test negative indicating proper sterilization conditions were met and the indicators should be positive after the survival cycles. After the cycles were complete, the container was opened and the indicator retrieved. The rapid sterility indicators were processed as described in Example 1, and the results obtained at 20 seconds or less after adding the indicator solution.

TABLE 3
One Container Reusable High Resistant Challenge Pack
134° C. Pre-Vacuum Steam Sterilizer
(Total steam exposure time, the first 8 minutes temperature is 60-30° C.)

| | Survival Time | Kill Time |
|---|---|---|
| | (9 Minutes) | (12 Minutes) |
| Rapid Sterility Indicator in | 10/10 | 0/10 |
| One Container Reusable | | |
| Challenge Pack | | |

The results in Table 3 were recorded as the number of positive over the number tested. When exposed to an extremely long survival cycle, including high temperatures, all indicators within the high resistant challenge pack tested positive. When exposed to the kill cycle time, all indicators tested negative. This table shows that the challenge pack design provided acceptable results. This resistant challenge pack has the unique feature of surviving very long cycles. It is actually testing the over-kill parameters built into the sterilizer. This also allows the user to do a validation in the standardized hospital cycles. The user can expose the indicator to the 3-4 pre-vacuums in a conventional autoclave and observe positive results. In the past, positive results of sterility indicators were only seen in BIER vessels or special research and development sterilizers that were able to perform one quick vacuum rather than the 3-4 long vacuums. This challenge pack also has the unique feature such that the user can reassemble the pack by placing a new indicator into the container, closing the container, and using it for another test.

### Example 4

### RSI Challenge Pack Testing in the Air Overpressure Cycle

Thirteen RSI units were processed with a mock load in a standard Air Overpressure cycle at nominal conditions of 122° C. for 31 minutes (cycle no. 1). Of the 13 samples, 5 exhibited wetting of the foam plug and 4 of the samples exhibited wetting and subsequent yellowing of the indicator tablet. The presence of excess moisture compromised the integrity of the indicator system and increased the potential for false-negative test results. In an effort to protect the RSI units from excess moisture, samples were either placed in preformed paper/plastic sterilization pouches, sealed in paper/plastic sterilization tubing, or wrapped in sterilizable surgical wrap and processed under the same cycle conditions (cycle no. 2).

TABLE 4

| | Cycle | | | | Wet | |
|---|---|---|---|---|---|---|
| Conditions | No. | Packaging¹ | Dry | Wet foam | tablet | Results² |
| Air | 1 | None | 8 | 5 | 4 | 0/13 |
| overpressure | | | | | | |
| Steam | | | | | | |
| Sterilizer | | | | | | |
| | | Pouch | n/a | n/a | n/a | n/a |
| | | Tubing | n/a | n/a | n/a | n/a |
| | | Wrap | n/a | n/a | n/a | n/a |
| | 2 | None | n/a | n/a | n/a | n/a |
| | | Pouch | 10 | 0 | 0 | 0/10 |
| | | Tubing | 10 | 0 | 0 | 0/10 |
| | | Wrap | 10 | 0 | 0 | 0/10 |

¹None = RSI unit without packaging.
Pouch = RSI unit placed within a Baxter 3.5” × 8” sterilization pouch.
Tubing = RSI unit placed within Roll Pack paper plastic sterilization tubing.
Wrap = RSI unit placed within sterilizable Surgical wrap.
²Data presented as the number of samples positive (non-sterile) per number tested.
n/a = not applicable.

As observed from the results in Table 4, when placed within an outer pouch, roll tubing or surgical wrap, neither the foam plug nor indicator tablet of the RSI unit were compromised by excess moisture during Air Overpressure steam sterilization.

### Example 5

### BIER Vessel Testing

To confirm that placement of the RSI unit into an outer paper/plastic pouch does not compromise its response to saturated steam sterilization, testing was conducted at 121.1° C. in a Joslyn steam BIER (Biological Indicator Evaluator Resistometer) vessel. A BIER vessel is the basis (physical standard) against which the performance of biological and chemical indicators are calibrated. The BIER vessel functions to provide reproducible exposure conditions with precise temperature control (±0.5° C.) and “square wave” cycle kinetics (time-to-temperature ≦15 seconds and time to exhaust to atmospheric pressure≦10 seconds).

TABLE 5

| Conditions | 5 min¹ | 9 min | 9.5 min | 10 min | 10.5 min | 15 min² |
|---|---|---|---|---|---|---|
| RSI alone | 10/10 | 8/10 | 4/10 | 2/10 | 1/10 | 0/10 |
| RSI in | 10/10 | 5/10 | 3/10 | 2/10 | 1/10 | 0/10 |
| pouch³ | | | | | | |

Data presented as the number of samples positive (non-sterile) per number tested.
¹Non-sterile conditions; all samples should give a positive response.
²Sterile conditions; all samples should give a negative response.
³Fisher 3.5″ × 5″ sterilization pouch (Cat # 01-812-50).

As observed from the results in Table 5, placement of the RSI unit within an outer pouch does not affect its response to saturated steam sterilization.

### Example 6

### Air Overpressure Steam Sterilization

To confirm that placement of the RSI unit into an outer paper/plastic pouch does not compromise its response to steam sterilization under Air Overpressure conditions, testing was performed under sublethal (all units non-sterile), fractional (some units non-sterile, some units sterile), and lethal (all units sterile) conditions. Testing was conducted with RSI units with and without an outer pouch. Conventional biological indicators were also included as part of the testing.

TABLE 6

| | | Sublethal | Fractional | Lethal |
|---|---|---|---|---|
| Conditions | Configuration | cycle¹ | cycle² | cycle³ |
| Air | RSI alone | 20/20 | 8/20 | 0/20 |
| Overpressure | | | | |
| Steam | | | | |
| Sterilizer | | | | |
| | RSI in pouch⁴ | 20/20 | 7/20 | 0/20 |
| | Attest alone⁵ | 20/20 | 3/20 | 0/20 |

Data is presented as the number of samples positive (non-sterile) per number tested.
¹Accumulated F₀ =<1. Cycle exposure setting of 112° C. for 1 min.
²F₀ = 8.0. Cycle exposure setting of 121° C. for 1 min.
³Accumulated F₀ = 42. Cycle exposure setting of 123° C. for 31 min.
⁴Fisher 3.5″ × 5″ sterilization pouch (Cat #01-812-50).
⁵3M Attest 1262 biological indicator for steam sterilization.

As observed from the results in Table 6, placement of the RSI unit within an outer pouch does not affect its ability to detect non-sterile conditions during Air Overpressure steam sterilization.

Other embodiments and uses of the invention will be apparent to those skilled in the art from consideration of the specification and practice of the invention disclosed herein. Although described in connection with the specific rapid sterility indicators disclosed herein, as would be clear to those of ordinary skill in the art, the pouch of the present invention can be used with other indicators, and is not limited to those described herein. All U.S. patents and other documents referenced herein, including U.S. Provisional patent Application Ser. No. 60/073,547, entitled Indicator Systems for Determination of Sterilization, are specifically incorporated by reference. It is intended that the specification and examples be considered exemplary only, with the true scope and spirit of the invention being indicated by the following claims.

We claim:

1. A test indicator device for determining the effectiveness of a sterilization procedure comprising:
a container having at least one opening that contains a fluid-transmissive barrier which communicates with an interior chamber that contains biological material used as an indicator of the destruction of living organisms by the sterilization procedure, and
a pouch in which the container is placed which is liquid impermeable and gas permeable under the sterilization procedure.

2. The device of claim 1 wherein the pouch contains a gas-permeable portion and a gas-impermeable portion.

3. The device of claim 2 wherein the gas-permeable portion is comprised of paper or a fibrous plastic sheet.

4. The device of claim 2 wherein the gas-impermeable portion is comprised of a transparent plastic film.

5. The device of claim 4 wherein the transparent plastic film comprises a plastic selected from the group consisting of nylon, polyester, polyvinyl chloride, and high-density polyethylene, or copolymers thereof.

6. The device of claim 1 wherein the pouch is adapted to peel open to deliver the test indicator for hands-on evaluation.

7. The device of claim 1 wherein the container has liquid impermeable and substantially non-absorptive walls.

8. A method for determining the effectiveness of a sterilization procedure comprised of:
placing a test indicator in a liquid impermeable and gas permeable pouch wherein the test indicator is comprised of a container having liquid impermeable and substantially non-absorptive walls and at least one opening communicating with an interior chamber, the interior chamber containing biological material used as an indicator of the destruction of living microorganisms by the sterilization procedure and a fluid-transmissive barrier positioned in the opening such that movement of gas between the environment surrounding the test indicator and the interior chamber occurs through the gas-transmissive barrier, and
exposing the test indicator and the pouch to the conditions of the sterilization procedure.

9. The method of claim 8 wherein the sterilization procedure is a gas over steam sterilization process.

10. The method of claim 8 wherein the pouch comprises a gas-permeable portion and a gas-impermeable portion.

11. The method of claim 10 wherein the gas-permeable portion is made of a paper or a fibrous plastic sheet.

12. The method of claim 10 wherein the gas-impermeable portion is made of plastic.

13. The method of claim 8 wherein the pouch is peeled open to deliver the test indicator for hands-on-evaluation.

14. A method for monitoring or determining the effectiveness of a sterilization procedure for contact lenses in blister packs which method employs a test indicator comprising a container having liquid impermeable and substantially non-absorptive walls and at least one opening communicating with an interior chamber, the chamber containing biological material used as an indicator of the destruction of living microorganisms by the sterilization procedure and a fluid-transmissive barrier positioned in the opening such that movement of fluid between the environment surrounding the test indicator and the interior chamber occurs through the fluid-transmissive barrier, and placing the test indicator inside a sealed pouch that is liquid impermeable and gas permeable under the conditions of gas over steam sterilization.
